(19) United States
(12) Patent Application Publication
(10) Pub. No.: US 2023/0414158 A1
(43) Pub. Date: Dec. 28, 2023

(54) SYSTEM AND A METHOD OF CAPTURING ELECTROENCEPHALOGRAMS (EEG) IN REAL-TIME

(71) Applicant: Benjamin Alan Erickson, Austin, TX (US)

(72) Inventor: Benjamin Alan Erickson, Austin, TX (US)

(21) Appl. No.: 18/213,387

(22) Filed: Jun. 23, 2023

Related U.S. Application Data
(60) Provisional application No. 63/355,048, filed on Jun. 23, 2022.

(51) Int. Cl. A61B 5/372; A61B 5/00

(52) U.S. Cl. CPC A61B 5/372; A61B 5/4064; A61B 5/165

(57) ABSTRACT

A system and method of capturing electroencephalograms (EEG) in real-time are disclosed. The system includes an EEG controller. A user wears or puts on the carrier. The carrier includes a casing for receiving the EEG controller, and includes a plate for connecting an image capturing unit. The EEG controller connects to cables having EEG sensors placed over the head of the user. The EEG sensors collect EEG signals and send them to the EEG controller via the cables. The image capturing unit captures images or video surrounding the user. The EEG controller captures data from the EEG sensors and the image capturing unit to monitor and evaluate the clinical status of the user.

The present application claims the benefit of U.S. Provisional Application No. 63/355,048, filed Jun. 23, 2022; all of which is incorporated herein by reference.

### FIELD OF INVENTION

The present subject matter generally relates to electroencephalogram (EEG) devices. More specifically, the present subject matter relates to a wireless system worn by a user, the wireless system including EEG sensors connected to the head of the user, and an image capturing unit to record the surroundings of the user.

### BACKGROUND OF INVENTION

It is known that biosignal data such as electroencephalograms (EEGs), electrocardiograms (ECGs) are very useful in monitoring a patient's condition. Monitoring of the biosignal data over long periods of time without disrupting the patient's routine provides high signal quality and/or presents the data in a useful and practical way to medical care specialists. The medical care specialists use the data to evaluate the clinical status of the user and provide the required care/treatment to the patient.

Several devices have been proposed in the past to detect signs and symptoms associated with seizure activity based on EEG signals. One such example is disclosed in a United States Publication No. 20190274571, entitled, “System and method for assessing brain function in real time after brain injury” (“the '571 Publication”). The '571 Publication discloses a method, and a system to perform the method, of assessing brain injuries in real time, the method including placing an EEG cap on a subject's head at a first location, processing diagnostic data including brain wave data using a first communication device at the first location, transmitting the diagnostic data to a second communication device located at a remote second location, and displaying the diagnostic data in real time on the second communication device.

Another example is disclosed in a U.S. Pat. No. 10,750,971, entitled, “Device for recording video-electroencephalograms” (“the '971 Patent”). The '971 Patent discloses a portable device for video electroencephalography. The device has a central portion to which a plurality of arcuate arms is directly or indirectly connected in a movable manner. The arms together define a helmet structure adapted to be worn on the head of a patient, arm is configured to allow mounting of one or more electrodes which are connected to an electronic central unit mounted on the helmet structure. The device also has a supporting member and a video camera mounted on the supporting member so as to face the helmet structure, said video camera. The arms have a first arm and a second arm which extend away from each other in a longitudinal direction (L), as well as a third arm and a fourth arm which extend away from each other in a front direction (F). The first and second arms are pivoted on the central portion about respective axes parallel to the front direction (F), and the third and fourth arms are pivoted about respective axes parallel to the longitudinal direction (L).

Another example is disclosed in a U.S. Pat. No. 10,874,356, entitled, “Wireless EEG headphones for cognitive tracking and neurofeedback” (“the '356 Patent”). The '356 Patent discloses a mobile device and methods for acquisition of biophysiological signals for the purpose of assessing mental states. The mobile device is embedded into wireless headset that comprises headphones. The mobile device comprises sensors for biophysiological signal acquisition, including, but not limited to, electroencephalographic (EEG) signals, pulse oximetry, heart rate, body temperature and electrodermal activity. Furthermore, the device also measures environmental factors, including, but not limited to, ambient light and sound from the environment. The mobile device administers sound and visual stimuli to the user. The said biophysiological signals, after being processed, are used to assess mental states of the user (emotional states and cognitive processes). Furthermore, the intensity of the said mental states can be maintained at the same level, enhanced or weakened or fully modified using visual or sound stimuli. The device is designed so that it maintains the ergonomics and compactness of the device, so that it can be generally accepted as an everyday consumer device.

Although the above disclosures are useful, they have few problems. For instance, the devices use conventional rigid structures around the head, bulky and are inflexible. Further, they use transducer interfaces consisting of multiple long wires, which reduce the patient's mobility during its use. Further, the existing devices are restricted to detecting motor activity and generalized tonic-clonic seizures. Furthermore, the existing devices only detect physiological changes or movements associated with seizures which can arise from many conditions other than seizures.

In addition, when a video camera is used to acquire ambulatory EEG, the patients move away from the camera. As a result, it is not possible to see the surrounding environment when the patient is on camera as confounds which need to be addressed for adequate assessment of patient events. There is also the phenomenon of patients who are unable to experience their typical events within the hospital setting in an epilepsy monitoring unit (EMU). Moreover, ambulatory video EEG monitoring (AVEM) may be of added benefit to patients who report seizures or events resembling epilepsy that occur in certain environments or with specific triggers that may not be replicated in the hospital setting.

Therefore, there is a need for at home or ambulatory EEG for characterization of the patient's events in the diagnosis of epilepsy. Further, there is a need for recording ambulatory EEG with video (AEEG)/ambulatory video EEG monitoring (AVEM) in the patient's every day setting that can reveal triggers that are only encountered in activities of daily living (ADL).

### SUMMARY

It is an object of the present subject matter to provide a system for capturing electroencephalograms (EEG) in real-time and that avoids the drawbacks of known devices.

It is an object of the present subject matter to provide a system that improves the quality of ambulatory video EEG (ambulatory video EEG monitoring (AVEM)) recordings through mounting of a camera on a device attached to the patient's body that captures eye movement, facial movement, arm movement, leg movement, automatisms, and the surrounding environment while remaining minimally obstructive to the patient's activities of daily living (ADL).

It is another object of the present subject matter to provide a system for capturing EEG signals and images/video of the surroundings of a user/patient wearing the system.

It is another object of the present subject matter to provide a system comprising a wearable device or carrier for housing a video camera and a controller for recording the EEG data.

It is yet another object of the present subject matter to provide a system that ensures there is full visibility of the patient's face and arms from a camera which is comfortably attached to a carrier worn by the patient. The camera capable of being operated remotely by a monitoring technologist who could check in at preset intervals rather than requiring 24-hour observation.

In order to achieve one or more objects, the present subject matter presents a system and a method of capturing electroencephalograms (EEG) in real-time. The system includes an EEG controller. A user wears or puts on the carrier. The carrier includes a casing for receiving the EEG controller, and includes a plate for connecting an image capturing unit. The EEG controller connects to cables having EEG sensors placed over the head of the user. The cables are affixed to the carrier (i.e., at straps of the carrier). The EEG sensors collect EEG signals and send them to the EEG controller via the cables. The image capturing unit captures images or video surrounding the user. The EEG controller captures data from the EEG sensors and the image capturing unit to monitor and evaluate the clinical status of the user.

In one aspect of the present subject matter, the system communicatively connects to a server via a network. The EEG controller transmits the data received from the EEG sensors and the image capturing unit to the server for doctors or physicians to evaluate the clinical status of the user.

In one advantageous feature of the present subject matter, the system transmits the data remotely to allow the doctors to evaluate the conditions of the user or patient at all times. This helps the doctors to provide immediate feedback and/or rapid evaluation of the potential seizure or any other activity in the brain.

In one advantageous feature of the present subject matter, the image capturing unit captures the images/video (360-degree view) of the user/patient. This helps the doctor to monitor his physical state and also the location of the user in a structure, say at home, office or hospital depending on the need.

In another advantageous feature of the present subject matter, the EEG controller is placed in a carrier, which is put over the shoulders/body of the user. Further, the cables are affixed to the carrier (i.e., at straps of the carrier). As a result, the user has freedom to move while reducing the risk of entanglement of the cables during use.

In yet another advantageous feature of the present subject matter, the placement of the EEG controller allows the user to perform his/her daily activities including sleeping without having to remove the carrier. Further, the carrier is easy to put on and remove by the user.

In yet another advantageous feature of the present subject matter, the casing made of a rigid material withstands the impact when the carrier or user falls on the ground and prevents the EEG controller from getting damaged.

In yet another advantageous feature of the present subject matter, the system eliminates the issues related to deterioration of EEG quality and enhances the quality of video. The system helps to increase the time the patient is on camera. Further, the system helps to increase yield of data useful in localization of seizures. Furthermore, the system helps to increase the ability to characterize seizure type by capturing the patient's face, arms, and legs from a single camera attached to the patient's body via the wearable. Particularly, the system captures video of the patient's face and arms while also recording audio. This is useful for localization of seizure onset and characterization of events.

In yet another advantageous feature of the present subject matter, the system detects electrical activity for an extended period through EEG. Concurrently to EEG “tracing”, the system records video of the patient for capturing clinical signs and symptoms which help in the localization of focal seizure onset and differential diagnosis. The data can be transmitted in real-time to an experienced clinician (neurologists, epileptologists) to diagnose seizures occurring in deep cerebral structures that are undetectable with scalp EEG recordings.

In yet another advantageous feature of the present subject matter, the system includes a carrier or bag that securely attaches to the patient at his/her torso. The carrier allows for free movement and the use of both arms without thought to the recording device. The carrier allows proper connection of all leads to the patient for the entirety of the recording of EEG signals. The carrier containing the casing houses an ambulatory EEG breakout box and routes all lead wires in a protected manner on the patient's body. Further, the system is designed to be comfortable enough that it can be worn for the majority of the full recording time including waking and sleeping hours, pose minimal interference with ADL, and be easily donned and doffed by the patient.

In yet another advantageous feature of the present subject matter, the system is provided in different sizes for use with children, adults alike with ease of use and comfort. In other words, the system presents a wearable carrier which is comfortable enough to wear throughout the night, adjustability to the patient's body, and is easy to use through intuitive design.

Features and advantages of the subject matter hereof will become more apparent in light of the following detailed description of selected embodiments, as illustrated in the accompanying FIGUREs. As will be realised, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

Further features and advantages of the present subject matter will become apparent from the following detailed description, taken in combination with the appended drawings, in which:

FIGS. 1A and 1B illustrate a side perspective and a front view, respectively of an environment in which a system for capturing electroencephalograms (EEG) implements, in accordance with one exemplary embodiment of the present subject matter;

FIG. 2 illustrates a perspective view of a carrier, in accordance with one embodiment of the present subject matter;

FIG. 3 illustrates a perspective view of the carrier having a second plate, in accordance with one embodiment of the present subject matter;

FIG. 4 illustrates a perspective view of a first plate having a receiving section, in accordance with one embodiment of the present subject matter;

FIGS. 5A and 5B illustrate a front and a rear view, respectively of an image capturing unit having a first lens and second lens, in accordance with one embodiment of the present subject matter;

FIG. 6 illustrates a top perspective view of a casing, in accordance with one embodiment of the present subject matter;

FIG. 7 illustrates an EEG controller placed in the casing, in accordance with one embodiment of the present subject matter; and

FIG. 8 illustrates the casing placed in the carrier, in accordance with one embodiment of the present subject matter;

FIG. 9 illustrates a block diagram of the system connecting to a server, in accordance with one embodiment of the present subject matter.

It will be noted that throughout the appended drawings, like features are identified by like reference numerals.

### DETAILED DESCRIPTION OF THE EMBODIMENTS

Before the present features and working principle of a system is described, it is to be understood that this subject matter is not limited to the particular system as described, since it may vary within the specification indicated. Various features of a system might be provided by introducing variations within the components/subcomponents disclosed herein. It is also to be understood that the terminology used in the description is for the purpose of describing the particular versions or embodiments only, and is not intended to limit the scope of the present subject matter, which will be limited only by the appended claims. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It should be understood that the present subject matter describes a system and method of capturing electroencephalograms (EEG) in real-time. The system includes an EEG controller. The carrier is worn by a user or patient. The carrier includes a casing for receiving the controller, and includes a plate for connecting an image capturing unit. The EEG controller includes cables having EEG sensors. The cables are placed around the head of the user to collect EEG signals. Here, the EEG cup electrodes are placed using the International 10/20 system of electrode placement and applied to the scalp using collodion. The image capturing unit captures images or video surrounding the user. The EEG controller captures data from the EEG sensors and the image capturing unit to monitor and evaluate the clinical status of the user.

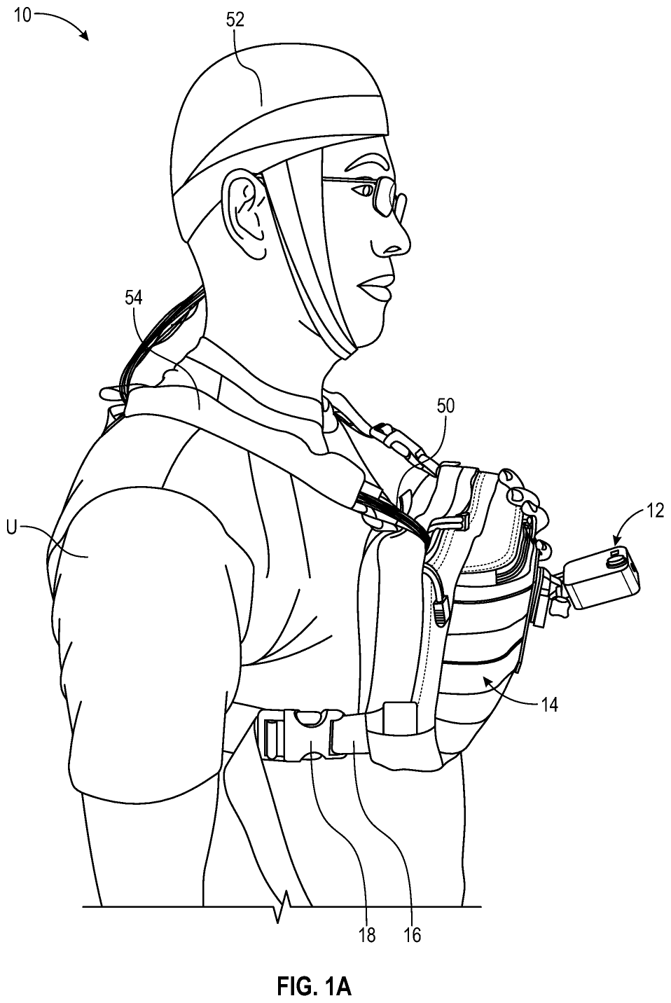

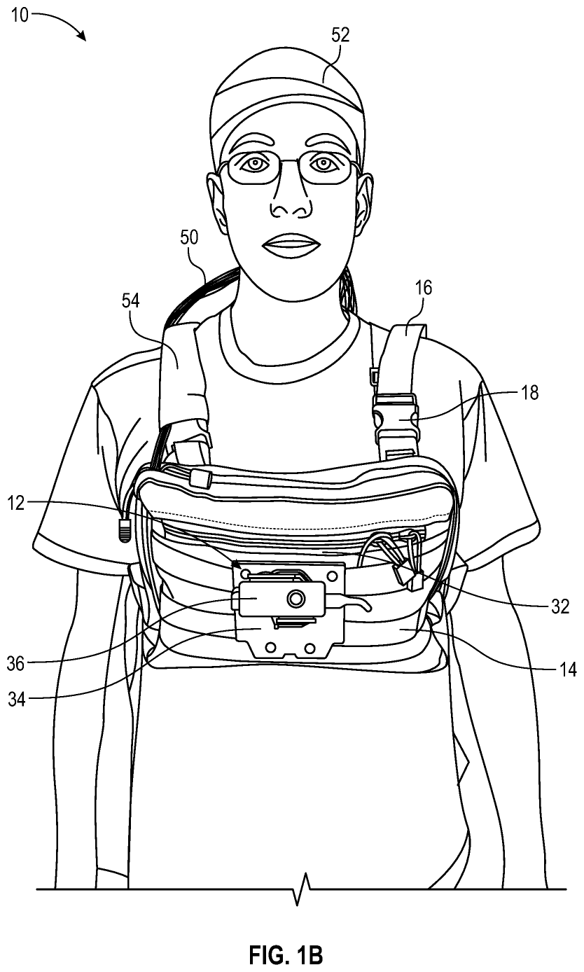

Various features and embodiments of a system for capturing electroencephalograms (EEG) in real-time are explained in conjunction with the description of FIGS. 1A-9.

The present subject matter discloses a system for capturing electroencephalograms (EEGs) in real-time. FIGS. 1A and 1B show a side perspective and a front view, respectively of an environment 10 in which system 12 implements, in accordance with one exemplary embodiment of the present subject matter. System 12 encompasses a carrier or bag or container or pack 14. A user U puts on carrier 14 over his/her shoulder and around his/her body/waist using straps 16 either at the front (chest area) or at the back (i.e., as a backpack). User U adjusts the length of straps 16 with the help of buckle 18.

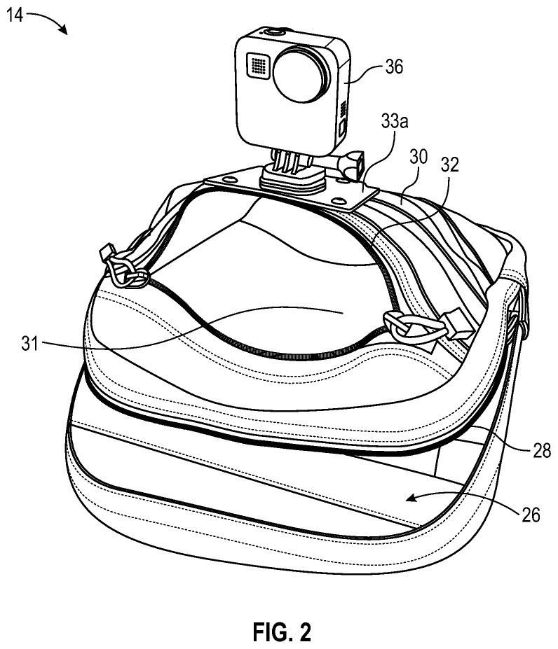

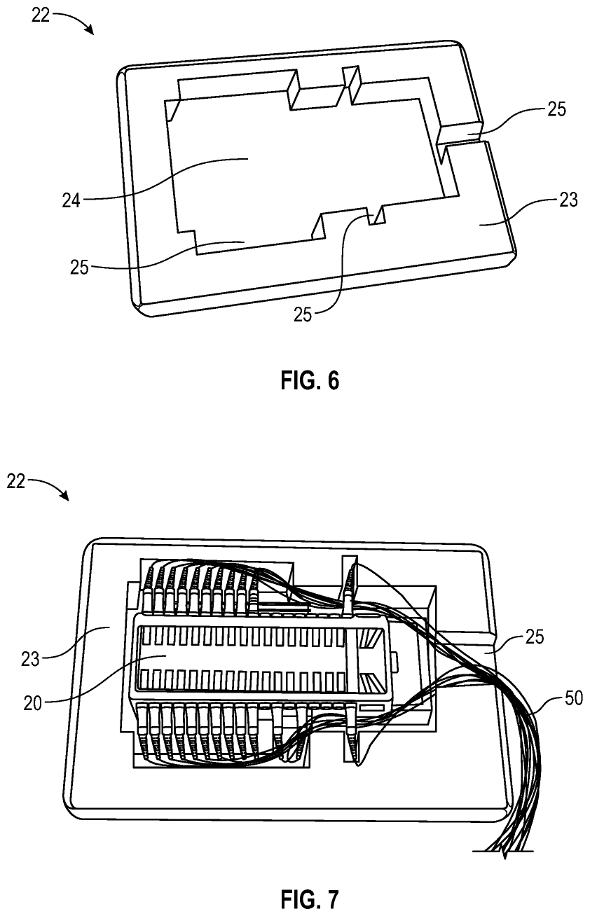

FIG. 2 shows a perspective view of carrier 14, in accordance with one embodiment of the present subject matter. Carrier 14 receives an EEG controller 20 in a casing 22. FIG. 6 shows a top perspective view of casing 22. Casing 22 indicates a container or structure holding EEG controller 20 inside carrier 14. Casing 22 is made of metal, plastic, ceramic, wood or any other suitable material. In one example, casing 20 includes a foam yoga block. Casing 22 is moulded or carved out of foam block to provide cushioning to controller 20. Casing 20 is designed based on the size and shape of controller 20. Casing 22 encompasses walls 23 defining a receiving section 24. Receiving section 24 indicates an opening formed within walls 23 for receiving EEG controller 20. Walls 23 include one or more cut sections 25. Cut sections 25 are formed by chipping away a portion of walls 23 adjacent to receiving section 24 and/or at predefined positions over the length and width of walls 23. In the present subject matter, EEG controller 20 inserts in receiving section 24 of casing 22, as shown in FIG. 7. In one example, casing 22 acts as a cushion member and protects controller 20 from potential impact.

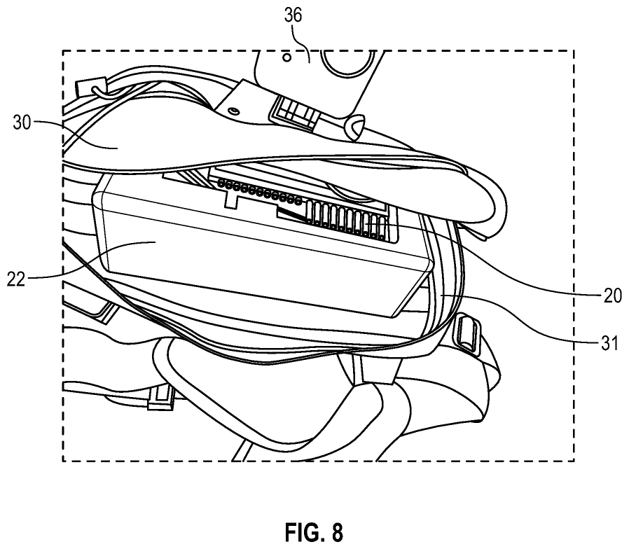

Carrier 14 includes first compartment 26, as shown in FIGS. 2 and 8. First compartment 26 indicates an opening or area surrounded by walls forming a closed structure. First compartment 26 includes a first closing mechanism 28 for allowing and restricting access to first compartment 26. Here, first closing mechanism 28 includes, but not limited to, a zipper, snaps, hook and loops, buttons, magnets or any other known and/or convenient fastening mechanisms for selecting engaging to close i.e., allowing or restricting access to first compartment 26. Carrier 14 encompasses sub-carrier 30 i.e., a smaller bag connecting adjacent to first compartment 26. Sub-carrier 30 defines a second compartment 31 i.e., an opening or area surrounded by walls forming a closed structure. Second compartment 31 includes a second closing mechanism 32 for allowing and restricting access to second compartment 31. Here, second closing mechanism 32 includes, but not limited to, a zipper, snaps, hook and loops, buttons, magnets or any other known and/or convenient fastening mechanisms for selecting engaging to close i.e., allowing or restricting access to second compartment 31.

Carrier or bag 14 bag attaches to the chest of the user with straps 16 emanating from a 10.5″×7.5″ rectangle of mesh material which seats opposite carrier 14 on the user's thoracic spine. Each strap 16 connects the back mesh to the chest bag with a quick release buckle 18. One strap 16 on each side wraps over each shoulder and one strap from each side wraps around the coastal region under each arm. Carrier 14 is made of suitable material and allows to wear outdoors during physical activity and is very comfortable when worn over clothing.

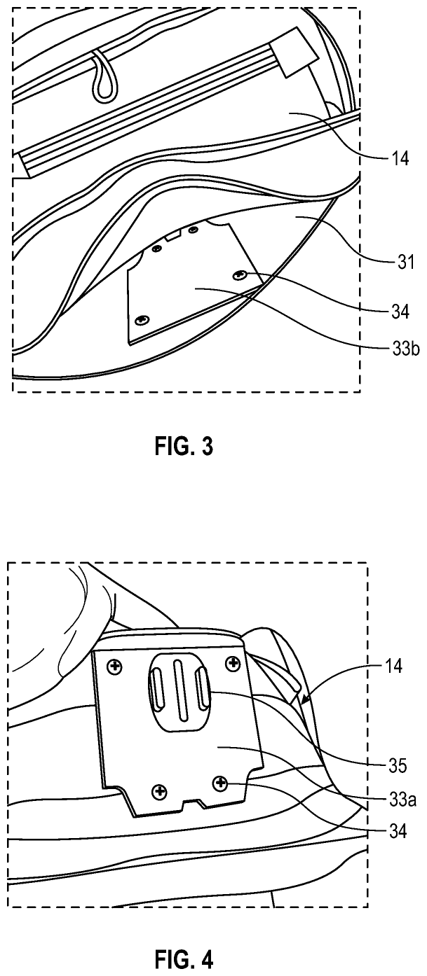

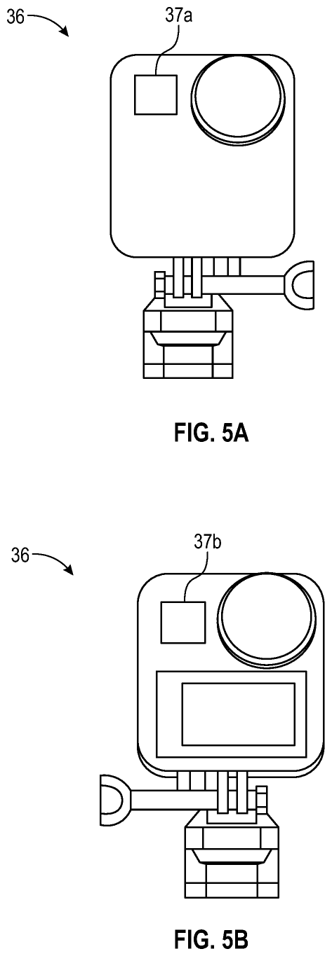

In one implementation, sub-carrier 30 includes a first plate 33a and a second plate 33b. First plate 33a faces outside of sub-carrier 30, as shown in FIG. 4. Second plate 33b faces inside sub-carrier 30/second compartment 31, as shown in FIG. 3. First plate 33a and second plate 33b connect with the help of connecting members or fasteners 34 as shown in FIGS. 3 and 4. In one embodiment, first plate 33a includes a receiving section 35 (FIG. 4) that slidably receives an image capturing unit 36. FIGS. 5A and 5B, show a front view and a rear view, respectively of image capturing unit 36. Image capturing unit 36 indicates a camera configured for capturing still images in its field of view or a series of frames (video). In one example, image capturing unit 36 includes a first lens 37a facing opposite to sub-carrier 30 such that image capturing unit 36 captures the surroundings in front of user U when user U wears carrier 14, as shown in FIG. 1A, for example. Further, image capturing unit 36 includes a second lens 37b pointing at the face of user U. In other words, image capturing unit 36 includes two lenses 37a and 37b, one facing the user's face and another facing the surroundings in front of user U, to capture the user's face and surroundings at the same time. Here, each of first lens 37a and second lens 37b includes a 180-degree fisheye camera. First lens 37a and second lens 37b are capable of recording simultaneously respective 180-degree views and stitch the edges of the captured images into a full 360-degree image which can be used in two different formats. In one example, first lens 37a and second lens 37b are capable of capturing and providing an immersive view allowing the viewer to pan through the entire 360-degree environment essentially bringing an entire room into view from a single camera in a single position. In another example Further, first lens 37a and second lens 37b are capable of capturing and providing a panoramic representation of the 360-degree version. In yet another embodiment, image capturing unit 36 includes a 360-degree camera capable of recording images/videos in every direction of a view.

Image capturing unit 36 is mounted in a such a way that both first lens 37a and second lens 37b would capture eye movement, facial movement, arm movement, leg movement, automatisms, and the surrounding environment while remaining minimally obstructive to the patient's activities of daily living (ADL). As such, first lens 37a and second lens 37b maintain user's visibility potentially 100% of the recording, and be minimally obstructive to ADL. In other words, the orientation and position of image capturing unit 36 is provided in such a way that one lens positions upwards (anatomical superior) towards the patients face and another lens points downward (anatomical inferior) towards the patient's feet. This provides full head to toe view of the patient with a minimal seam between the upward and downward cameras which makes no difference in the ability to view the entire user at all times.

Carrier 14 is provided to hold controller 20 with enough extra room for padding to protect the patient and the equipment. FIG. 8 shows the feature of carrier 14 holding controller 20 in casing 22. Optionally, carrier 14 also has an outer pouch that is large enough to hold battery 48 with extra room for housing on-board computing or Wi-Fi hotspot devices.

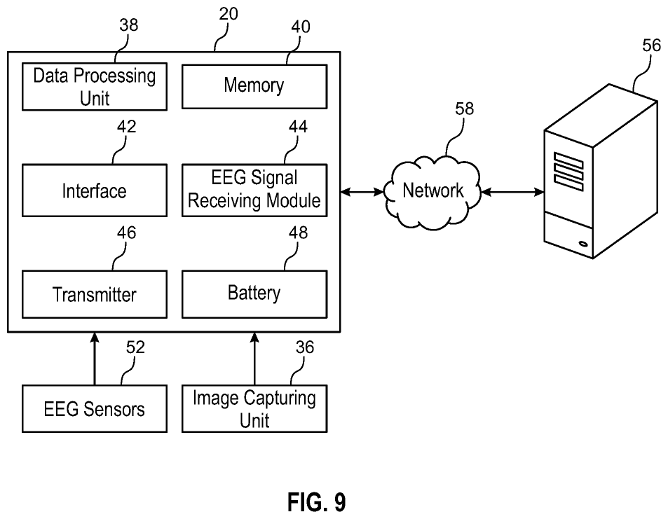

FIG. 9 shows a block diagram of controller 20, in accordance with one embodiment of the present subject matter. Controller 22 includes a data processing unit 38. Data processing unit 38 indicates a processor or a central processing unit (CPU), a graphics processing unit (GPU) or both. Controller 22 includes memory 40 capable of storing instructions. The instructions reside, completely or at least partially, within the memory 40 and/or within data processing unit 38 during execution thereof. The instructions are further transmitted or received over a network 58 via transceiver 46 utilizing any one of a number of well-known transfer protocols.

Controller 22 encompasses interface 42 such as hardware and/or software devices/applications used for controller 20. Controller 22 includes EEG signal receiving module 44. EEG signal receiving module 44 communicatively connects to EEG sensors 52 positioned over the head of user U. EEG signal receiving module 44 receives EEG signals from EEG sensors 52 via cables 50.

Controller 22 encompasses transceiver 46 for sending or receiving instructions/data from other devices such as a server 56 via network 58. Controller 22 further encompasses battery 48 for powering electronic components in controller 22. Server 56 indicates a central server operated by a hospital, medical institution, etc., for storing and processing the health data of user U. Network 58 includes a wireless network, a wired network or a combination thereof. Network 58 can be implemented as one of the different types of networks, such as intranet, local area network (LAN), wide area network (WAN), the internet, and the like. The network 58 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 58 may include a variety of network devices, including routers, bridges, servers, computing devices, storage devices, and the like.

In one example, controller 20 includes one or more buttons or toggles (not shown) to turn ON/OFF image capturing unit 36 or transmitter 46.

In the present subject matter, cables 50 extend from controller 20 and position over straps 16 and connect around head area of user U. Here, cables 50 affix to straps 16 with the help of connecting member 54 such as adhesive strip, adhesive tape, fabric or any other suitable material. Connecting member 54 prevents cables or lead wires 50 from falling apart and remains in position at straps 16, as shown in FIG. 1B, for example. Cables 50 include EEG sensors or EEG electrodes 52 to collect brain signals and send EEG signals to data processing unit 38 via EEG signal receiving module 44. EEG sensors 52 include smaller disk-shaped EEG electrodes positioned over the head of user U.

The presently disclosed system 12 provides EEG tracing of high quality throughout the recording. In operation, data processing unit 38 receives EEG signals from EEG sensors 52. Data processing unit 38 amplifies the EEG signals to reduce or remove a common-mode neural field potential signal present in EEG sensors or electrodes 52, and outputs a resulting differential signal indicative of a neural action potential. Concurrently or consecutively, data processing unit 38 receives the images or video captured from image capturing unit 36. In one example, data processing unit 38 processes the data received from EEG sensors 52 and image capturing unit 36 to check clinical status of user U. Alternatively, data processing unit 38 transmits the data received from EEG sensors 52 and image capturing unit 36 to server 56 for doctors or physicians to evaluate the clinical status of user U. In the absence of internet connection, data processing unit 38 processes the data received from EEG sensors 52 and image capturing unit 36 and stores in memory 40. Once the internet connection is active, data processing unit 38 transmits the data to server 56. The doctors use a user device (not shown) such as a mobile phone, a laptop, etc., to access server 56 and view/interact with user U. Alternatively, the doctors operate image capturing unit 36 remotely to check EEG signals of user U in at pre-set intervals depending on the need.

Based on the above, it is evident that the presently disclosed system records video with a 360-degree camera mounted to a bag or carrier on the patient's chest while simultaneously recording AEEG for clinical seizure presentation. The system helps to collect eye movement; hand, arm, and shoulder movement; feet and legs; and the entire room surrounding the patient which may reveal potential seizure triggers which would not be visible from a conventional camera's field of view all at the same time from a single camera.

The presently disclosed system allows the patient to go wherever they want in the house and yet allow the doctors (neurologists, epileptologists) see the patient's face, arms, hands, etc. in great detail. Also, the system allows the camera to move/capture images in all directions, which provides an added advantage of being able to assess the whole seizure. In a normal EEG, the system helps the doctors to provide patients and families clear descriptions of the events. In epileptic events, the system helps with lateralization and localization.

The carrier holding the controller and image capturing unit allows the patient to sit, recline, and sleep flat on his/her back with the carrier securely in place. This reduces deterioration of lead connection quality by reducing or eliminating tension the controller can apply to the connection of the electrode cups to the scalp. By eliminating this potential tension, skin breakdown also reduces caused due to mechanical injury from EEG lead cups being pulled and released repeatedly and wearing of the skin at the contact point over the seventy-two-hours of recording typical in AEEG studies.

A person skilled in the art appreciates that the system may come in a variety of sizes depending on the need and comfort of the user. Further, different materials in addition to or instead of materials described herein may also be used and such implementations may be construed to be within the scope of the present subject matter. Further, many changes in the design and placement of components may take place without deviating from the scope of the presently disclosed system.

In the above description, numerous specific details are set forth such as examples of some embodiments, specific components, devices, methods, in order to provide a thorough understanding of embodiments of the present subject matter. It will be apparent to a person of ordinary skill in the art that these specific details need not be employed, and should not be construed to limit the scope of the subject matter.

In the development of any actual implementation, numerous implementation-specific decisions must be made to achieve the developer's specific goals, such as compliance with system-related and business-related constraints. Such a development effort might be complex and time-consuming, but may nevertheless be a routine undertaking of design, fabrication, and manufacture for those of ordinary skill. Hence as various changes could be made in the above constructions without departing from the scope of the subject matter, it is intended that all matter contained in the above description or shown in the accompanying drawings shall be interpreted as illustrative and not in a limiting sense.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. It is contemplated that additional embodiments are within the spirit and true scope of the disclosed subject matter.

I claim:

1. I claim all of the above subject matter.
